(19) United States
(12) Patent Application Publication
(10) Pub. No.: US 2019/0010419 A1
(43) Pub. Date: Jan. 10, 2019

(54) PROCESS FOR EXTRACTING LIPIDS FOR USE IN PRODUCTION OF BIOFUELS

(71) Applicant: DSM IP Assets B.V., TE Heerlen (NL)

(72) Inventors: Kirk APT, Ellicott City, MD (US); William BARCLAY, Boulder, CO (US); Micah BLAZER, San Diego, CA (US); Jacob BORDEN, Lake Charles, LA (US); Adam Burja, Columbia, MD (US); Daniel DONG, Woodstock, MD (US); Armando DURAZO, San Diego, CA (US); Jean-Charles DUMENIL, Little Chaufront (GB); Arthur EDGE, Naperville, IL (US); Jon HANSEN, Chandler, AZ (US); Alexandra HOFLER, San Diego, CA (US); David JEFFERS, Woking Surray Guziable (GB); Chris LYON, San Diego, CA (US); Vidya PAI, Rockville, MD (US); Joseph William PFEIFER, III, Westminster, MD (US); Martin J. SELLERS, Workingham (GB); Ginger SHANK, Winchester, KY (US); Justin STEGE, San Diego, CA (US)

(21) Appl. No.: 16/130,575

(22) Filed: Sep. 13, 2018

Related U.S. Application Data
(63) Continuation of application No. 15/106,752, filed on Jun. 20, 2016.
(60) Provisional application No. 61/918,850, filed on Dec. 20, 2013.

(51) Int. Cl. C11B 1/02; C10L 1/02; C12N 9/26; C12N 1/06; C12N 9/24; C11B 1/12; C11B 1/10; C12P 7/64

(52) U.S. Cl. CPC C11B 1/025; C12Y 302/01001; C10L 1/02; C10L 2200/0469; C10L 2290/26; C12N 9/2414; Y02E 50/343; C12P 7/6463; C12N 9/2411; C12N 1/066; C12N 1/063; C12N 9/2488; C11B 1/12; C11B 1/10

(57) ABSTRACT

Methods and systems used to extract lipids suitable in production of biofuels from a fermentation broth may include using heat to pre-treat the fermentation broth in order to more easily extract a product from oleaginous microorganisms in the broth. Additionally or alternatively, a combination of enzymes including amylase, 1-4 mannosidase, and 1-3 mannosidase may be used to break down cell walls of the oleaginous microorganisms. Residual broth water may be recycled and used as imbibition water for washing a process feedstock to extract sugar.

### CROSS-REFERENCE TO RELATED APPLICATIONS

This application claims benefit of U.S. provisional patent application Ser. No. 61/918,850 filed Dec. 20, 2013.

### NAMES OF THE PARTIES TO A JOINT RESEARCH AGREEMENT

For purposes of prior art determination, a joint research agreement was executed between BP Biofuels UK Limited and Martek Biosciences Corporation on Dec. 18, 2008 in the field of biofuels. Also for purposes of prior art determination, a joint development agreement was executed between BP Biofuels UK Limited and Martek Biosciences Corporation on Aug. 7, 2009 in the field of biofuels. Also for purposes of prior art determination, a joint development agreement was executed between BP Biofuels UK Limited and DSM Biobased Products and Services B.V. on Sep. 1, 2012 in the field of biofuels.

### TECHNICAL FIELD

The invention relates to methods and systems directed to extracting materials for biofuels production. Aspects of the invention relate to extracting materials from oleaginous microorganisms.

### BACKGROUND

A number of technologies for converting feedstocks into biofuels have been developed. However, even with these advances in technology, there remains a need and a desire to improve economic viability for conversion of renewable carbon sources to fuels.

Vegetable-oil-derived biofuel may have benefits, such as being renewable, biodegradable, nontoxic, and, in certain cases, containing neither sulfur nor aromatics. But one potential disadvantage of vegetable-oil-derived biofuel is high cost, most of which is due to the cost of the vegetable oil feedstock. Therefore, the economic aspect of biofuel production has been at least somewhat limited by the cost of the vegetable oil raw materials, as well as the limited supply of the vegetable oil raw materials.

Lipids for use in nutritional products can be produced in microorganisms. Manufacturing a lipid in algae, for example, may include growing the algae, drying it, and extracting the intracellular lipids from it. Extracting material from within the microorganism can be difficult.

Similarly, yeasts, including oleaginous yeast, have polysaccharide cell walls to protect them from environmental stresses, such as shear forces, osmotic imbalances, desiccation, predators, and the like. The protective cell wall can make it difficult to harvest intracellular metabolites, such as lipids, in oleaginous yeast that can be converted into biofuel.

Converting sugar to biofuel using heterotrophic organisms is possible with an aqueous or solvent extraction section, in which parts of the organisms are dissolved in water or another solvent, thus enabling the product lipids to be removed and recovered directly from a fermentation broth. Product may be recovered from internal compartments of the oleaginous organism by combinations of mechanical, thermal, osmotic, and enzymatic forces, resulting in a multi-phase product stream consisting of light lipids, delipidated biomass, and aqueous residue and other cell residue. Once-through processing often results in a considerable waste and/or co-product stream(s).

There is a need and a desire for methods and systems for extracting materials from oleaginous microorganisms that result in a high yield of material extracted using non-solvent/aqueous extraction techniques. There is a further need and desire for methods and systems for extracting materials from oleaginous microorganisms that recycle residual process streams, rather than relying on once-through processing.

### SUMMARY

The invention relates to methods and systems for extracting materials from oleaginous microorganisms, as well as methods and systems for producing biofuels from the extracted materials.

According to certain embodiments, temperature may be used as a pre-treatment step to improve the extraction yield of product from an oleaginous organism. More particularly, a method of extracting lipids suitable in production of biofuels from a whole fermentation broth may include pre-treating the whole fermentation broth by heating the broth to a temperature greater than about 90° C., such as between about 90° C. and about 150° C., or between about 100° C. and about 150° C., or between about 110° C. and about 150° C., or between about 120° C. and about 150° C., or between about 130° C. and about 150° C., wherein the broth contains oleaginous microorganisms, and subsequently extracting a product from the oleaginous microorganisms. The whole fermentation broth may be heated for more than about 3 hours. In certain embodiments, time spent by the whole fermentation broth containing the oleaginous microorganisms between 45° C. and 80° C. may be minimized by heating the whole fermentation broth containing the oleaginous microorganisms from 45° C. to 80° C. in less than 60 minutes. Additionally or alternatively, the whole fermentation broth may be heated at an average rate between about 0.1 and about 80 degrees Celsius per minute. In this process, pH of the whole fermentation broth may be adjusted by adding either an acid or a base.

In further embodiments, the whole fermentation broth may be cooled to greater than about 60° C., or greater than about 70° C., or greater than about 80° C., or greater than about 85° C., or greater than about 90° C. to allow further isothermal (constant temperature) processing, such as by applying mechanical disruption. The whole fermentation broth may be cooled at an average rate between about 1 and about 80 degrees Celsius per minute. The whole fermentation broth may be agitated at an impeller tip speed between about 10 cm per second and about 240 cm per second. Following the heating, the whole fermentation broth may be dried.

In certain embodiments, during the pre-treating, a pressure between about 10 psi and about 150 psi, or between about 20 psi and about 150 psi, or between about 30 psi and about 150 psi, or between about 50 psi and about 150 psi may be maintained in a system containing the whole fermentation broth.

During the pre-treating, salts may be present in a system containing the whole fermentation broth, resulting in an ionic strength estimated between about 0.01 M and about 2.0 M in the system. The whole fermentation broth may include a crude sugar source and/or a water source associated with salts and ions at a concentration greater than 0.05 g/L. The salts and ions may include Na, K, Ca, Mg, Zn, Mo, Cu, Mn, chlorides, sulfates, phosphates, nitrates, and combinations thereof. These salts and ions may build up to a concentration of 0.5 to 40 g/L. Additionally, the salts and ions that are already present may help with recovery of an oil phase by promoting coalescence, flocculation, density change, and/or destabilizing the emulsion formed when the product is released from the oleaginous microorganisms in mechanical and/or electrostatic coalescers.

Methods herein may further include subjecting the oleaginous microorganisms to lysis, resulting in an oil body and cellular debris particle size distribution in which at least 80%, or at least 95%, of a volume of released product oil bodies and cellular debris have a size greater than 0.1 um in diameter. Additionally, the oil and cell debris droplets or bodies may be recovered as a continuous phase by mixing at an impeller tip speed of greater than 120 cm/s.

After breaking down the oleaginous cell walls, intracellular metabolites including lipids, for example, may be harvested from the oleaginous cell walls. The intracellular metabolites may be converted into biofuel, such as bio-derived diesel. An aqueous extraction effluent remaining after harvesting the intracellular metabolites may be recycled. The recycled extraction water may be used as imbibition water for washing a process feedstock to extract sugar.

Following the pre-treating, the fermentation broth may be depressurized and cooled to concentrate solids in the broth prior to further processing. Additionally or alternatively, following the pre-treating, evaporators or dryers could be included to generate a concentrated wet broth or a dry mixture with cells. A solvent may be added to the dry cells or to the lysed concentrated fermentation broth to form a mixture. The solvent may include hexane, dodecane, decane, diesel, one or more alcohols, or combinations thereof. The mixture of the lysed fermentation broth and the solvent may be agitated to contact and extract oil from the oleaginous microorganisms. The solvent and the oil may be separated from the lysed fermentation broth, such as by using a centrifuge. The solvent and the oil may be reacted to convert at least a portion of the oil into a fuel component. Furthermore, the solvent and a remainder of the oil may be converted into a fuel comprising a biofuel. The spent broth may be used as fertilizer for crops, animal feed, yeast extract, yeast hydrolysate, or a source of carbon/nutrients.

In certain embodiments, the whole fermentation broth containing the oleaginous microorganisms may include a sugar feedstock. The whole fermentation broth containing the oleaginous microorganisms and the sugar feedstock may include about 50 to about 250 grams of lipid per liter of fermenter broth, about 0 to about 50 grams of sugar per liter of fermenter broth, about 0 to about 40 grams of salt per liter of fermenter broth, and about 10 to about 100 grams of lipid-free dry biomass per liter of fermenter broth.

According to some embodiments, as part of the pre-treatment, the method may further include pasteurizing a whole fermentation broth containing the oleaginous microorganisms, such as by heating the whole fermentation broth to about 40° C. to about 80° C. for about 1 minute up to about 3 hours. In contrast, during the pre-treatment heating, the whole fermentation broth may be held at a temperature between about 90° C. and about 150° C., or between about 100° C. and about 150° C., or between about 110° C. and about 150° C., or between about 120° C. and about 150° C., or between about 130° C. and about 150° C. for about 30 minutes to about 18 hours, or more than 3 hours to about 18 hours, or more than 3 hours to about 8 hours. The whole fermentation broth may be stirred during the heating interval. An acid, a base, or both an acid and a base may be added to the whole fermentation broth.

The whole fermentation broth may be passed through a bead mill, orifice plate, high shear mixer, or other shear or mechanical disruption device once, twice, or more. In certain embodiments, the whole fermentation broth may be stirred in a vessel at about 70° C. to about 100° C. optionally including reflux for about 1 to about 60 hours. Additionally, a salt such as NaCl, KCl, K₂SO₄, or Na₂SO₄ may be added to the whole fermentation broth in the vessel or alternatively may be produced in situ, for example, by adding NaOH or KOH, plus H₂SO₄. Up to about 2% by weight of the salt may be added, for example. An acid or a base may be added to adjust a pH of the whole fermentation broth in the vessel to between about 3 and about 11. The heat generated from the combination of acids and bases listed above could also contribute to reducing the energy required for heating the broth. The lipids may be separated from the aqueous fermentation broth through an appropriate solid-liquid-liquid separation scheme that may include one or more steps such as gravity separation, hydrocyclones, filters, and/or centrifuges. Oil that is less than 20% free fatty acids may be separated from the whole fermentation broth through centrifugation. This method of extracting lipids suitable in the production of microbial oil may result in an oil that is artificially lower in metals, as the aqueous extraction process concentrates the metals in fermentation broth as compared to the oil by a ratio of at least 2. The method may further include recycling the biomass solids with the residual broth water.

The oleaginous microorganisms may include at least 40% by weight fat. For example, the oleaginous microorganisms may be oleaginous yeast cells.

According to certain embodiments, a combination of enzymes including amylase, 1-4 mannosidase, and 1-3 mannosidase may be used to break down oleaginous cell walls of the oleaginous microorganisms. The combination of enzymes may further include at least one auxiliary enzyme, namely sulfatase, protease, chitinase, or any combinations of these enzymes. The amylase may be specific for alpha 1-4 linked glucose. The combination of enzymes may include between about 5% and about 30% by weight amylase, between about 5% and about 45% by weight 1-4 mannosidase, between about 5% and about 45% by weight 1-3 mannosidase, or any combination of these parameters. The enzyme combination may also include at least one auxiliary enzyme, such as sulfatase, protease, chitinase, or any combination of these enzymes. The enzyme combination may be used with Sporidiobolus pararoseus MK29404. As mentioned, after breaking down the oleaginous cell walls, intracellular metabolites including lipids, for example, may be harvested from the oleaginous cell walls.

According to certain embodiments, a method of extracting lipids suitable in production of biofuels from a whole fermentation broth may include applying a combination of enzymes to the whole fermentation broth containing oleaginous microorganisms to break down cell walls of the oleaginous microorganisms, wherein the enzymes include amylase, 1-4 mannosidase, and 1-3 mannosidase, and subsequently extracting a product from the oleaginous microorganisms. The combination of enzymes may further include at least one auxiliary enzyme such as sulfatase, protease, chitinase, or any combination of these enzymes. The amylase may be specific for alpha 1-4 linked glucose. The combination of enzymes may include between about 5% and about 30% by weight amylase, between about 5% and about 45% by weight 1-4 mannosidase, between about 5% and about 45% by weight 1-3 mannosidase, or any combination of these parameters.

The method may further include harvesting intracellular metabolites, such as lipids, from the oleaginous microorganisms after breaking down the cell walls. The intracellular metabolites may be converted into biofuel, such as bio-derived diesel. Additionally, an aqueous extraction effluent remaining after harvesting the intracellular metabolites may be recycled. The recycled extraction water may be used as imbibition water for washing a process feedstock to extract sugar.

According to certain embodiments, a method of extracting lipids suitable in production of biofuels from an aqueous fermentation broth may include extracting lipids from the aqueous fermentation broth, wherein the broth contains oleaginous microorganisms or sugarcane, or both oleaginous microorganisms and sugarcane, leaving biomass solids and residual broth water, and using the residual broth water as imbibition water for washing a process feedstock to extract sugar. The method may further include pasteurizing the aqueous fermentation broth, such as by heating the aqueous fermentation broth to about 40° C. to about 80° C. for about 1 minute up to about 3 hours. In certain embodiments, the method may include heating the aqueous fermentation broth to a temperature between about 90° C. and about 150° C., or between about 100° C. and about 150° C., or between about 110° C. and about 150° C., or between about 120° C. and about 150° C., or between about 130° C. and about 150° C. and holding the broth within the selected range for about 30 minutes to about 18 hours, or more than 3 hours to about 18 hours, or more than 3 hours to about 8 hours. The aqueous fermentation broth may be stirred during the heating interval. An acid, a base, or both an acid and a base may be added to the aqueous fermentation broth. The aqueous fermentation broth may be passed through a bead mill or other mechanical disruption device once, twice, or more.

### BRIEF DESCRIPTION OF THE DRAWINGS

The accompanying drawings, which are incorporated in and constitute a part of this specification, illustrate embodiments of the invention and, together with the description, serve to explain the features, advantages, and principles of the invention. In the drawings:

FIG. 1 is a process flow diagram illustrating one embodiment of an aqueous extraction process using temperature pre-treatment and including the production of a yeast extract.

FIG. 2 is a process flow diagram illustrating one embodiment of an integrated sugar-to-diesel process including recycle.

FIG. 3 is a process flow diagram illustrating an aqueous extraction process used in Example 2.

FIG. 4 is a graphical representation of particle size distribution of released oil and cell debris following lysis in Example 3.

FIG. 5 is a graphical representation of particle size distribution of oil and cell debris following oil product recovery in Example 3.

### DETAILED DESCRIPTION

The invention provides methods and systems for extracting materials from oleaginous microorganisms, as well as methods and systems for producing biofuels from the extracted materials. Production of biofuels from microorganisms may have many advantages over production of biofuels from plants (including oilseeds), such as short life cycle, less labor requirement, independence of season and climate, and easier scale-up.

As described in greater detail below, pre-treatment of fermentation broth before oil extraction by directly heating the broth to relatively high temperatures can increase the amount of oil extracted from oleaginous microorganisms via thermal degradation of the cell wall structure such that permeability is increased and oil can diffuse more readily. Additionally or alternatively, a combination of enzymes including amylase, 1-4 mannosidase, and 1-3 mannosidase may be used to break down oleaginous cell walls of the oleaginous microorganisms. In any of the methods described herein, the aqueous extraction effluent remaining after lipid removal may be recycled to the front-end sugar recovery operations.

As used herein, the terms “pre-treat” and “pre-treatment” refer to process steps that are carried out on a microorganism prior to physically separating any materials from within the microorganism.

As used herein, the term “renewable material” preferably refers to a substance and/or an item that has been at least partially derived from a source and/or a process capable of being replaced at least in part by natural ecological cycles and/or resources. Renewable materials may broadly include, for example, chemicals, chemical intermediates, solvents, adhesives, lubricants, monomers, oligomers, polymers, biofuels, biofuel intermediates, biogasoline, biogasoline blendstocks, biodiesel, green diesel, renewable diesel, biodiesel blend stocks, biodistillates, biochar, biocoke, biological oils, renewable building materials, and/or the like. As a more specific example, the renewable material may include, without being limited to, any one or more of the following: methane, ethanol, n-butanol, isobutanol, 2-butanol, fatty alcohols, isobutene, isoprenoids, triglycerides, lipids, fatty acids, lactic acid, acetic acid, propanediol, butanediol. According to certain embodiments, the renewable material may include one or more biofuel components. For example, the renewable material may include an alcohol, such as ethanol, butanol, or isobutanol, or lipids. In certain embodiments, the renewable material can be derived from a living organism, such as algae, bacteria, fungi, and/or the like. According to certain embodiments, the renewable material is a lipid, such as fatty acids with a carbon chain length profile at least somewhat similar to rapeseed oil.

The term “biofuel” preferably refers to components and/or streams suitable for use as a fuel and/or a combustion source derived at least in part from renewable sources. The biofuel can be sustainably produced and/or have reduced and/or no net carbon emissions (total carbon lifecycle) to the atmosphere, such as when compared to fossil fuels. According to certain embodiments, renewable sources can exclude materials mined or drilled, such as from the underground. In certain embodiments, renewable sources can include single cell organisms, multi-cell organisms, plants, fungi, bacteria, algae, cultivated crops, non-cultivated crops, timber, and/or the like.

According to certain embodiments, the renewable sources include microorganisms. Biofuels can be suitable for use as transportation fuels, such as for use in land vehicles, marine vehicles, aviation vehicles, and/or the like. More particularly, the biofuels may include gasoline, diesel, jet fuel, kerosene, and/or the like. Biofuels can be suitable for use in power generation, such as raising steam, exchanging energy with a suitable heat transfer media, generating syngas, generating hydrogen, making electricity, and/or the like. According to certain embodiments, the biofuel is a blend of biodiesel and petroleum diesel.

The terms “biodiesel” and “bio-derived diesel,” as used herein, are used interchangeably and refer to components or streams suitable for direct use and/or blending into a diesel pool and/or a cetane supply derived from renewable sources. Suitable biodiesel molecules can include fatty acid esters. Biodiesel can be used in compression ignition engines, such as automotive diesel internal combustion engines, truck heavy duty diesel engines, and/or the like. In the alternative, the biodiesel can also be used in gas turbines, heaters, boilers, and/or the like. According to certain embodiments, the biodiesel and/or biodiesel blends meet or comply with industrially accepted fuel standards, such as B5, B7, B10, B15, B20, B40, B60, B80, B99.9, B100, and/or the like.

The term “lipid,” as used herein, refers to oils, fats, waxes, greases, cholesterol, glycerides, steroids, phosphatides, cerebrosides, fatty acids, fatty acid related compounds, derived compounds, other oily substances, and/or the like. Lipids typically include a relatively high energy content, such as on a weight basis.

The term “microorganism,” as used herein, refers to a microscopic organism, which may be a single cell (unicellular), a cell cluster, or a multicellular relatively complex organism. Microorganisms can include algae, fungi (including yeast), bacteria, cyanobacteria, protozoa, and/or the like.

In one embodiment, the microorganism can be a single cell member of the fungal kingdom, such as a yeast, for example. Examples of oleaginous fungi that can be used include, but are not limited to, Rhodotorula ingeniosa or Sporidiobolus pararoseus, as well as members of the following genera: Aspergillus, Candida, Cryptococcus, Debaromyces, Endomycopsis, Fusarium, Geotrichum, Hyphopichia, Lipomyces, Mucor, Penicillium, Pichia, Pseudozyma, Rhizopus, Rhodotorula, Rodosporidium, Sporobolomyces, Starmerella, Torulaspora, Trichosporon, Wickerhamomyces, Yarrowia, Zygoascus, and Zygolipomyces. More particularly, the oleaginous fungi may include, for example, any of the following: Apiotrichum curvatum, Candida apicola, Candida bombicola, Candida oleophila, Candida sp., Candida tropicalis, Cryptococcus albidus, Cryptococcus curvatus, Cryptococcus terricolus, Debaromyces hansenii, Endomycopsis vernalis, Geotrichum carabidarum, Geotrichum cucujoidarum, Geotrichum histendarum, Geotrichum silvicola, Geotrichum vulgare, Hyphopichia burtonii, Lipomyces lipofer, Lipomyces orentalis, Lipomyces starkeyi, Lipomyces tetrasporous, Pichia mexicana, Rhodosporidium sphaerocarpum, Rhodosporidium toruloides, Rhodotorula sp., Rhodotorula aurantiaca, Rhodotorula dairenensis, Rhodotorula diffluens, Rhodotorula glutinus, Rhodotorula glutinis, Rhodotorula gracilis, Rhodotorula graminis, Rhodotorula minuta, Rhodotorula mucilaginosa, Rhodotorula mucilaginosa, Rhodolorula rubra, Rhodotorula terpenoidalis, Rhodotorula toruloides, Sporobolomyces alborubescens, Starmerella bombicola, Torulaspora delbruekii, Torulaspora pretoriensis, Torulopsis lipofera, Toruposis sp., Trichosporon behrend, Trichosporon brassicae, Trichosporon capitatum, Trichosporon cutaneum, Trichosporon domesticum. Trichosporon laibachii, Trichosporon loubieri, Trichosporon montevideense, Trichosporon pullulans, Trichosporon sp., Wickerhamomyces canadensis, Yarrowia lipolytica, Zygoascus meyerae, and Zygolipomyces lactosus.

The extraction methods described herein may be applied to essentially any oleaginous microorganism. The microorganism can operate, function, and/or live under any suitable conditions, such as anaerobically, aerobically, photosynthetically, heterotrophically, and/or the like. According to certain embodiments, the yeast may be cultured heterotrophically in the presence of air.

The term “oleaginous,” as used herein, refers to oil bearing, oil containing and/or producing oils, lipids, fats, and/or other oil-like substances. Oleaginous may include organisms that produce at least about 20 percent by weight of oils, at least about 30 percent by weight of oils, at least about 40 percent by weight oils, at least about 50 percent by weight oils, at least about 60 percent by weight oils, at least about 70 percent by weight oils, at least about 80 percent by weight oils, and/or the like of the total weight of the organism. Oleaginous may refer to a microorganism during culturing, lipid accumulation, at harvest conditions, and/or the like.

Lipids suitable for use in production of biofuels may be extracted from a whole fermentation broth containing oil-rich microbial cells of oleaginous microorganisms. According to certain embodiments, the whole fermentation broth may include a sugar feedstock. For example, the whole fermentation broth may include about 50 to about 250 grams of lipids per liter of fermenter broth, about 0 to about 50 grams of sugar per liter of fermenter broth, about 0 to about 40 grams of salt per liter of fermenter broth, and about 10 to about 100 grams of lipid-free dry biomass per liter of fermenter broth. The oleaginous microorganisms may include at least 40% by weight fat, or between about 40% and about 80% by weight fat, or between about 50% and about 75% by weight fat, in certain embodiments.

Prior to the thermal pre-treatment, the whole fermentation broth may be pasteurized to inactivate cellular enzymes and to eliminate the viability of the production organism to prevent replication upon storage. Pasteurization also provides adequate control measures to minimize damage to products of interest, in this case by also inactivating the lipases. The pasteurization may be carried out by heating the whole fermentation broth to less than about 90° C., such as between about 40° C. and about 80° C., for less than 3 hours, such as between about 1 minute and just under 3 hours.

As mentioned, the amount of oil extracted may be increased by pre-treating the whole fermentation broth with heat. Pre-treatment of the whole fermentation broth includes thermal treatment with concurrent changes in process pH, which is intended to effect a thermo-chemical change in the cell wall composition. More particularly, by directly heating the broth to a temperature greater than 90° C., such as between about 90° C. and about 150° C., or between about 91° C. and about 150° C., or between about 100° C. and about 150° C., or between about 110° C. and about 150° C., or between about 120° C. and about 150° C., or between about 130° C. and about 150° C., for greater than 3 hours, the cell wall structure undergoes thermal degradation, which increases permeability of the cell wall, thus enabling oil to diffuse more readily during subsequent extraction of a product from the oleaginous microorganisms. The exact nature of change depends on the cell wall chemistry of the production strain. For oleaginous yeasts, release of carbohydrates (monomers) comprising the cell walls has been observed as a result of the pre-treatment. For example, holding the whole fermentation broth at 121° C. for just over 3 hours to about 8 hours with gentle stirring using an impeller may provide 80-85% extractability by increasing the porosity of the cells. While not essential, the system may be vented to the atmosphere to facilitate concentration of fermentation broth from 80% down to 70% water content via evaporation. To minimize lipolytic activity, it may be desirable to minimize the time spent by the whole fermentation broth containing the oleaginous microorganisms between 45° C. and 80° C. This minimization may be achieved by heating the whole fermentation broth containing the oleaginous microorganisms from 45° C. to 80° C. in less than 60 minutes. In certain embodiments, the whole fermentation broth may be heated at an average rate between about 0.1 and about 80 degrees Celsius per minute.

During pre-treatment, the pH of the whole fermentation broth may also be adjusted, either by adding an acid or a base. For example, by first adding an acid and then a base, this treatment may result in the early release of oil. Through the use of reagents and other aids, the pH may be adjusted to any level within a range of about 0.5 to 14, using acids, bases, salts, or any combination of acids, bases, or salts. For example, an acid may be added to adjust the pH to between about 3.0 and about 6.0. As another example, a base may be added to adjust the pH to between about 8.0 and about 10.5. In certain embodiments, during the pre-treatment salts may be present in a system containing the whole fermentation broth, resulting in an ionic strength estimated between about 0.01 M and about 2.0 M in the system. The pre-treatment step is the most aggressive prolonged thermal treatment step in the process and a majority of the chemical reactions occur during this period. The subsequent mechanical lysis step releases the oil into a relatively inert, unreactive environment. Fermentation broth that has been through the pre-treatment may be coalesced within less than 8 hours, suitably with 4 hours of additional heating at over 90° C. and mixing alone. In comparison, broth that has been through pasteurization alone may require extensive additional mixing, such as more than 8 hours of additional mixing, at under 90° C. to allow separation of an oil phase.

According to some embodiments, the whole fermentation broth may include a crude sugar source associated with salts and ions at a concentration greater than about 0.05 g/L. As used herein, the term “crude sugar” refers to sugar extracts containing one or more disaccharides or monosaccharides derived from complex renewable feedstock (including cane sugar, sweet sorghum, and sugar beets) or concentrated forms of sugar extracts including sugar juice, raw juice, thick juice, and molasses. Crude sugar can contain any combination of disaccharides and monosaccharides at greater than 15 wt % up to 95 wt %, with water, salts, minerals, feedstock residue, and complex biomass forming the remainder. Crude sugar may alternatively be described as containing from 60% to 99% as a ratio of sugar monomers to other solids in the dry matter. The other non-sugar components of the dry matter may include salts, minerals, feedstock residue, and complex biomass.

These salts and ions associated with the crude sugar source may include Na, K, Ca, Mg, Zn, Cu, Mn, Fe, Co, and chlorides, sulfates, phosphates, nitrates, and combinations thereof. These salts and ions are thus introduced at concentrations beyond those required for fermentative growth of the microorganisms. The salts and ions may accumulate to a concentration of 0.5 to 40 g/L, for example. Of particular uniqueness are potassium and calcium, which may accumulate to higher concentrations than most other elements and are different from usual fermentation broth media. These unique properties may facilitate the separation of lipid and water phases. More particularly, the concentration of potassium is suitably higher than the concentration of sodium. In certain embodiments, the concentration of calcium may be greater than 1 g/L. In certain embodiments, the concentration of potassium may be greater than 2.5 g/L. At the built-up concentration, the introduced salts and ions help with recovery of the oil phase by a coalescence when the product oil is released from the microbial cells. Additionally, at the built-up concentration, the introduced salts and ions eliminate the need for addition of salts and ions often required to help with recovery of the oil phase by coalescence, i.e., inducers or demulsifiers. Thus, the fermentation broth may maintain the same ion ratios or concentrations during the coalescence stage, as well as during other downstream steps, as during the pre-treatment. For example, the fermentation broth may have a concentration of salts and ions of 0.5 to 40 g/L during coalescence.

Salts of additives to improve extraction may be added to the fermentation broth, or to the wash water for the sugar source, or to both the fermentation broth and the wash water for the sugar source. Along with the salts and ions, nutrient feeds, crude or purified nutrient sources, nitrogen or carbon, crude or partially refined sugar sources, and/or different water sources may also be added to the fermentation media.

When carrying out the extraction method on a fermentation broth that includes a crude sugar, a crude oil may be recovered that is lower in metals and inorganic elements, such as Na, K, P, Ca, Mg, Zn, and the like, compared to extraction techniques that utilize whole dried biomass and/or solvents to recover crude oil.

Also during the pre-treatment, a pressure between about 10 psi and about 150 psi, or between about 20 psi and about 150 psi, or between about 30 psi and about 150 psi, or between about 50 psi and about 150 psi may be maintained in a system containing the whole fermentation broth. This effective temperature and pressure may be lower if the system is held under a vacuum using steam jet ejectors.

Following the heating, the whole fermentation broth may be cooled or dried, or both cooled and dried, to allow further isothermal (constant temperature) processing. More particularly, “isothermal processing” refers herein to processing without the need for additional heating or cooling. With respect to the cooling and/or drying, for example, the whole fermentation broth may be cooled to greater than about 60° C., or greater than about 70° C., or greater than about 80° C., or greater than about 85° C., or greater than about 90° C. The fermentation broth may also be depressurized, in combination with the cooling, to concentrate solids in the broth prior to further processing. Further processing may include the application of mechanical disruption, using such devices as a bead mill, a homogenizer, an orifice plate, a high-shear mixer, a press, an extruder, pressure disruption, wet milling, dry milling, or other shear or mechanical disruption device for one pass, two passes, or more. For example, two passes through a bead mill may provide greater than 90% extractability. The further addition of acid may facilitate coalescence. The whole fermentation broth may be cooled at an average rate between about 0.2 and about 80, or between about 0.2 and about 1 degree Celsius per minute, for example. Additionally or alternatively, a flash evaporator may be used to concentrate the solids in the broth.

To provide additional agitation, the whole fermentation broth may be stirred in a vessel at a temperature between about 70° C. and about 100° C. optionally including reflux for between about 1 and about 60 hours, thus providing 60 to 85% oil recovery, for example. If desired, the whole fermentation broth may be held agitated at an impeller tip speed between about 10 and about 300 cm per second, or between about 120 and about 240 cm per second. This agitation may be carried out using any advantageous combination of radial and axial flow impellers, such as Rushton or marine impellers, for example. Optionally, further temperature adjustments, pH adjustments, salt addition, or any combination of these actions may be made during the agitation. For example, up to about 2% by weight of salt, such as NaCl, KCl, K₂SO₄, or Na₂SO₄, may be added to the whole fermentation broth in the vessel or alternatively may be produced in situ, for example, by adding NaOH or KOH, plus H₂SO₄. As another example, an acid or a base may be added to adjust a pH of the whole fermentation broth in the vessel to between about 3 and about 11. The heat generated from the combination of acids and bases listed above could also contribute to reducing the energy required for heating the broth.

As an additional pre-treatment step, the oleaginous microorganisms may be subjected to lysis, resulting in an oil body and cellular debris particle size distribution in which at least 80%, or at least 95%, of a volume of released product oil bodies and cellular debris have a size greater than 0.1 um in diameter, with the diameter being the greatest distance across the droplet, particle, or body. The diameter may be measured using a Particle Size Analyzer, available from Malvern Instruments Ltd of Worcestershire, UK. More particularly, the thermal pre-treatment assists in the lysis, which frees the oil once the biomass is digested away. Due to this particle size distribution, the oil and cell debris droplets may be easily recovered as a continuous phase through simple mixing coalescence steps at an impeller tip speed of greater than 120 cm per second, on a 3-inch (7.62 cm) Rushton type impeller, for instance. The coalesced lipid may result in a coalesced lipid particle size distribution in which at least 80%, or at least 95%, of a volume of coalesced lipids have a size greater than about 40 um in diameter, for example.

A solvent may be added to either the dry cells or the lysed fermentation broth, following the heating, to form a mixture. Examples of suitable solvents include hexane, dodecane, decane, diesel, alcohols, polar solvents, non-polar solvents, and combinations thereof. The mixture may then be agitated to allow the solvent to contact and extract the oil from the whole cells of the oleaginous microorganisms. After a suitable period of time, the stream can be separated, such as by using a centrifuge, settling tank, cyclone, or any combination of these techniques, to separate the solvent and the oil from the fermentation broth. The solvent and the oil stream may then be reacted to convert the oil into a fuel component prior to converting the solvent and a remainder of the oil into a fuel comprising a biofuel. This method of extracting lipids suitable in the production of microbial oil results in an oil that is artificially lower in metals, as the aqueous extraction process concentrates the metals in fermentation broth as compared to the oil by a ratio of at least 2.

Residual biomeal or spent broth resulting from the thermal pre-treatment described herein may include hydrolysed cell wall polysaccharides and proteins in aqueous solution, including media and generated salts, as well as de-solventized cell wall debris, either lysed or unlysed. The residual delipidated biomeal or spent broth may be used as fertilizer for crops, animal feed, yeast extract, or a source of carbon/nutrients, for example. More particularly, due to the high levels of potassium in the fermentation broth, the spent broth may be recycled as a potassium source in the form of a fertilizer for sugar fields or other crops. Using an aqueous process, the residual biomeal may be in better form for these other potential uses compared to residual biomeal resulting from non-aqueous processes.

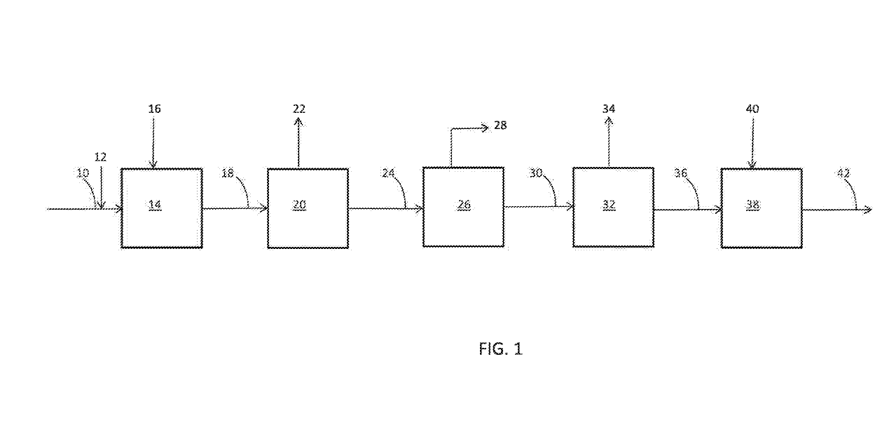

FIG. 1 illustrates one example of an aqueous extraction process using temperature pre-treatment and including the production of a yeast extract. The process begins with a fermentation broth 10, to which a base 12 may be added (optionally). While the fermentation broth 10 is heated in a vessel 14 to 121° C. and held at this temperature for approximately 8 hours, an acid 16 may be added (optionally). Following the heat treatment, the pre-treated broth 18 is then cooled (such as by flash-cooling, for example) to 60° C. in a cooling device 20 and in the process, water vapor 22 is released. Concentrated broth 24 is then transferred to a centrifuge 26, which separates the broth 24 into an oil stream 28 and an aqueous extraction residual stream 30. Other types of separation techniques, such as a settling tank or a cyclone, may also be used either alone or in combination with one another. The aqueous extraction residual stream 30 is directed to a pressurizer 32 (or evaporator), from which water 34 from the pressure is released and a yeast cake 36 is formed and forwarded to a hydrolyser 38 into which an acid 40 is added. The result is a hydrolysed yeast cake 42.

Microorganisms on which the processes herein may be carried out include, but are not limited to, algae, fungi, and bacteria. For example, a suitable fungi may include oleaginous yeast, such as those belonging to the genus Rhodotorula, Pseudozyma, or Sporidiobolus.

According to certain embodiments, the yeast belongs to the genus Sporidiobolus pararoseus. In a specific embodiment, the disclosed microorganism is the microorganism corresponding to ATCC Deposit No. PTA-12508 (Strain MK29404 (Dry1-13J)). In another specific embodiment, the microorganism is the microorganism corresponding to ATCC Deposit No. PTA-12509 (Strain MK29404 (Dry1-182J)). In another specific embodiment, the microorganism is the microorganism corresponding to ATCC Deposit No. PTA-12510 (Strain MK29404 (Dry1-173N)). In another specific embodiment, the microorganism is the microorganism corresponding to ATCC Deposit No. PTA-12511 (Strain MK29404 (Dry55)). In another specific embodiment, the microorganism is the microorganism corresponding to ATCC Deposit No. PTA-12512 (Strain MK29404 (Dry41)). In another specific embodiment, the microorganism is the microorganism corresponding to ATCC Deposit No. PTA-12513 (Strain MK29404 (Dry1)). In another specific embodiment, the microorganism is the microorganism corresponding to ATCC Deposit No. PTA-12515 (Strain MK29404 (Dry1-147D)). In another specific embodiment, the microorganism is the microorganism corresponding to ATCC Deposit No. PTA-12516 (Strain MK29404 (Dry1-72D)).

Yeasts have polysaccharide cell walls to protect them from environmental stresses, such as shear forces, osmotic imbalances, predators, and the like. The protective cell wall can make it difficult to harvest intracellular metabolites, such as lipids in oleaginous yeast that can be converted into biofuel.

Glycosidic enzymes are useful in breaking down polysaccharides, and thus for degrading yeast cell walls. Glycosidic enzymes are often active on the specific sugar monomers within a polysaccharide, and the specific linkages between monomer sugars. For instance, glycosidic enzymes can differentiate between α-1-4 linked glucose (amylose) and β-1-4 linked glucose (cellulose). However, yeast are not all composed of identical polysaccharides but rather differ widely with respect to the types and ratios of saccharide monomers and the types of linkages between monomers.

Consequently, the mixture of glycosidic enzymes that are optimal for cell wall degradation is dependent on the organism. One particular oleaginous yeast used in converting sugar-to-diesel, Sporidiobolus pararoseus MK29404Dry1, has a particularly novel cell wall structure. A common structural linkage in many yeast is β-1-3 glucan. However, MK29404Dry1 showed only a little 1-3 linked glucose, and instead α-1-4 glucose was the major sugar linkage. Another common component of yeast cell walls is mannan, which is often composed of 1-6 linked mannose monomers. In contrast, MK29404dryl contains very little 1-6-mannose, but rather contains both 1-3 and 1-4 linked mannose.

Because of the particular composition of the MK29404Dry1 yeast cell wall, a specific combination of enzymes is needed to effectively degrade the microbial cell wall. It has been discovered that a combination of enzymes including amylase, 1-4 mannosidase, and 1-3 mannosidase is particularly effective in breaking down oleaginous cell walls of oleaginous microorganisms that include MK29404Dry1. In particular, amylases specific for α-1-4 linked glucose are especially effective. For example, the combination of enzymes may include between about 5% and about 30%, or between about 6% and about 25%, or between about 7% and about 20% by weight amylase; between about 5% and about 45%, or between about 10% and about 35%, or between about 15% and about 30% by weight 1-4 mannosidase; and between about 5% and about 45%, or between about 10% and about 35%, or between about 15% and about 30% by weight 1-3 mannosidase.

The combination of enzymes may also include one or more auxiliary enzymes, such as proteases, sulfatases, chitinases, or any combination of these enzymes to improve enzyme performance and lipid recovery.

Either prior to or subsequent to using the combination of enzymes to break down the oleaginous cell walls within a whole fermentation broth containing oleaginous microorganisms, the whole fermentation broth may be thermally pre-treated as described above. More particularly, the broth may be heated to a temperature between about 90° C. and about 150° C. for more than 3 hours.

After using the combination of enzymes to break down the oleaginous cell walls within a whole fermentation broth containing oleaginous microorganisms, intracellular metabolites may be harvested from the oleaginous cell walls. The intracellular metabolites suitably contain lipids. The extracted lipids may be used in the production of biofuels, such as bio-derived diesel.

More particularly, as described in greater detail above, a solvent, such as hexane, dodecane, decane, diesel, alcohols, or any combination of these solvents, may be added to the dry cells or lysed fermentation broth to form a mixture. The mixture of the broth and the solvent may be agitated to contact and extract oil from the oleaginous yeast cells. The solvent and the oil may subsequently be separated from the broth, such as by using a centrifuge. The solvent and the oil may be reacted to convert at least a portion of the oil into a fuel component. The solvent and a remainder of the oil may be converted into a fuel, namely a biofuel. The spent broth may be used as fertilizer for crops, animal feed, yeast extract, yeast hydrolysate, or a source of carbon/nutrients.

As described in greater detail below, any aqueous extraction effluent remaining after harvesting the intracellular metabolites may be recycled. For example, the recycled extraction water may be used as imbibition water for washing a process feedstock to extract sugar.

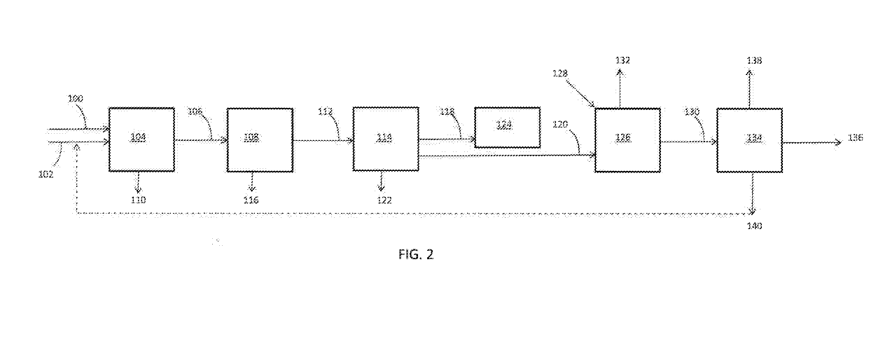

FIG. 2 is an integrated sugar-to-diesel flowsheet showing how aqueous extraction effluent remaining after lipid removal is recycled to the front-end sugar recovery operations. More particularly, recycled extraction water is used as imbibition water for washing the process feedstock to extract sugar. Such integrations are beneficial because a greater yield on feed materials is realized, as well as a reduction in waste management capital and processing costs.

While it is always of interest to recycle waste streams, the key is identifying the proper recycle point within the flowsheet that maximizes recovery value, while also accounting for how recycling affects the dynamics and optimum operation of the integrated flowsheet.

As described above, sugar may be converted to biofuel, including diesel for example, using heterotrophic organisms with an aqueous extraction section, whereby the product lipids are removed and recovered directly from the aqueous fermentation broth. Product is recovered from internal compartments of the oleaginous organism by combinations of thermal, mechanical, osmotic, and enzymatic forces, resulting in a multi-phase product stream that contains less dense lipids, residual broth water, and delipidated biomass. As illustrated in FIG. 2, the residual broth water can be recycled and used as imbibition water for washing a process feedstock to extract sugar.

The flowsheet in FIG. 2 shows sugarcane 100 and imbibition water 102 fed to a mill 104. From the mill 104, a sugar solution 106 is fed to a treatment device 108, while bagasse 110 is separated out. From the treatment device 108, an MEV (multi-effect evaporator) feed 112 is sent to evaporators 114, while mud 116 is separated out. From the evaporators 114, a vapor/gas stream 118 is fed to a seed fermentation device 124, while a concentrated sugar stream 120 is fed to a main fermentation device 126, and water 122 is separated out. Along with the concentrated sugar stream 120, air 128 is also added to the main fermentation device 126. From the main fermentation device 126, broth 130 is fed to an aqueous extraction device 134, while water vapor and CO₂ 132 are released. From the aqueous extraction device 134, a product oil 136 is separated out, evaporated water 138 is released, and waste water 140 is recycled into the imbibition water stream 102. Table 1 shows sample flow magnitudes for major streams and components in the sugar-to-diesel flowsheet of FIG. 2. Based on the data in Table 1, an imibibation water reduction of 40% is calculated, with this reduction attributable to recycling the waste water. Additionally, a solid supplement to bagasse of 5% is calculated, also attributable to recycling the waste water.

TABLE 1
Flow Magnitudes for Major Streams and Components
in the Sugar-to-Diesel Flowsheet

| | Sugar | Water | Oil | Bagasse | Biomeal | Total |
|---|---|---|---|---|---|---|
| Sugarcane | 140 | 660 | | 185 | | 985 |
| Imbib water | | 300 | | | | 300 |
| Bagasse | 1.4 | 185 | | 185 | | 371 |
| Sugar soln | 138.6 | 775 | | | | 914 |
| Mud | | 10 | | | | 10 |
| MEV feed | 138.6 | 765 | | | | 904 |
| VG steam | | 140 | | | | 140 |
| Conc sugar | 138.6 | 156 | | | | 295 |
| Water | | 469 | | | | 469 |
| Water vap | | 20.3 | | | | 20 |
| Broth | 1.386 | 174 | 35 | | 8.75 | 220 |
| Waste water | 1.386 | 103.56 | 5.25 | | 8.75 | 119 |
| Product oil | | 0.92 | 29.75 | | | 31 |
| Evap water | | 70 | | | | 70 |

Recycling the waste water as a portion of the imbibition water generates multiple unexpected benefits, including:
1. Recovery of organic carbon that can be further converted to product (see benefit 5)
2. At least partial recovery of organic and inorganic nutrients from non-lipid biomass and reduced first intent nutrient feed (e.g. ammonia)
3. Separation of unrecovered non-lipid biomass from solution by comingling with bagasse
4. Additional boiler feed and energy generation by comingling bagasse and unrecovered, non-lipid biomass
5. Reduced stringency on fermentation operation allowing for greater organic carbon slippage and recovery by recycle (see benefit 1)
6. Optimization of sugar streams 106 and 120 for use as specific feed streams to the seed and main fermenters
7. Reduction in fresh water demand by using recycle water for imbibition
8. Reduction in capital and operating costs for waste treatment

The recycle streams may be implemented in the previously-described methods to improve the recovery and conversion of key constituents and improve overall efficiency. For example, in a method of extracting lipids suitable in production of biofuels from an aqueous fermentation broth, wherein the broth contains oleaginous microorganisms or sugarcane, or both oleaginous microorganisms and sugarcane, the aqueous fermentation broth may be pasteurized, such as by heating the aqueous fermentation broth to about 40° C. to about 80° C. for about 1 minute up to almost 3 hours. The aqueous fermentation broth may be thermally pre-treated by heating the broth at a temperature between about 90° C. and about 150° C., or between about 100° C. and about 150° C., or between about 110° C. and about 150° C., or between about 120° C. and about 150° C., or between about 130° C. and about 150° C. for about 30 minutes to about 18 hours, or more than 3 hours to about 18 hours, or more than 3 hours to about 8 hours. The aqueous fermentation broth may be stirred during the heating interval. An acid, a base, or both an acid and a base may be added to the aqueous fermentation broth. The aqueous fermentation broth may be passed through a bead mill or other mechanical device at least once, or at least twice, or more. The aqueous fermentation broth may be stirred in a vessel at about 70° C. to about 100° C. or under reflux for about 1 to about 60 hours. A salt, such as up to about 2% by weight of the salt, such as NaCl, KCl, K₂SO₄, or Na₂SO₄, may be added to the aqueous fermentation broth in the vessel or alternatively may be produced in situ, for example, by adding NaOH or KOH, plus H₂SO₄. An acid or a base may be added to adjust a pH of the aqueous fermentation broth in the vessel to between about 3 and about 11. The lipids may be separated from the aqueous fermentation broth through an appropriate solid-liquid-liquid separation scheme that may include one or more steps such as gravity separation, hydrocyclones, filters, and/or centrifuges, leaving biomass solids and residual broth water. The residual broth water can be used as imbibition water for washing the process feedstock to extract sugar. Additionally, the biomass solids can be recycled with the residual broth water.

The lipids may be converted into a biofuel through the use of hydrotreating or transesterification, for example.

According to certain embodiments, the invention may be directed to a manufacturing facility for producing biofuels. According to certain embodiments, the manufacturing facility may include a lipid extraction unit. Additionally, the manufacturing facility may include a thermal pre-treatment unit. In certain embodiments, the manufacturing facility may include equipment that enables recycle of residual broth water.

According to certain embodiments, the invention may be directed to a renewable material or a biofuel, or both a renewable material and a biofuel, made according to any of the methods described herein.

According to certain embodiments, the methods described herein may result in an increase in the oil extraction yield of the microorganism. For example, the method may result in an increase in the oil extraction yield of the microorganism of at least about 10 weight percent. According to certain embodiments, the increase in oil extraction yield may be at least about 10 weight percent, at least about 15 weight percent, or at least about 20 weight percent.

### EXAMPLES

One metric used to characterize the performance of the described microorganisms is fatty acid extractability, or FAE. The FAE of any of the microorganisms according to the disclosure can be calculated according to the following formula:

$$\frac{𝓁}{b \times {C}_{biomass}} = 1 - ( \frac{{c}_{biomeal} \times ( 100 - {C}_{biomass} )}{{C}_{biomass} \times ( 100 - {c}_{biomeal} )} )$$

wherein b is the total biomass after cell rupture, typically measured in grams;

C_{biomass} is the percentage of FAME prior to cell rupture, wherein C_{biomass} is calculated as total grams FAME over total grams biomass; the term “FAME,” as used herein, refers to a fatty acid methyl ester;

c_{biomeal} is the percentage of FAME after cell rupture, wherein c_{biomeal} is calculated as total grams FAME over total grams biomeal; and

l is the total mass of oil after cell rupture, but prior to the oil recovery step, typically measured in grams. Obtaining these values from the microorganism or fermentation broth is within the ability of one of ordinary skill in the art.

According to some embodiments, the methods described herein may result in an increase in the oil or fatty acid extractability index of the microorganism. For example, the method may result in an increase in the FAE index of the microorganism of at least about 10 weight percent.

In certain embodiments, following oil recovery with hexane, the mass of the oil is measured (L). Also measured is the FAME after oil recovery with hexane. In some embodiments, vacuum evaporation, as is known in the art, is performed on the sample prior to FAME measurement.

The extraction yield of any of the microorganisms according to the disclosure can be calculated according to the following formula:

$$100 \times \frac{L \times {C}_{oil}}{B \times {C}_{biomass}}$$

wherein B is the total biomass prior to cell rupture, typically measured in grams;

C_{biomass} is the percentage of FAME prior to cell rupture, wherein C_{biomass} is calculated as total grams FAME over total grams biomass;

Cₒᵢₗ is the percentage of FAME after cell rupture and oil recovery, wherein Cₒᵢₗ is calculated by total grams FAME over total grams oil; and

L is the total mass of oil after cell rupture and oil recovery, typically measured in grams. Obtaining these measurements from the microorganism or fermentation broth is within the ability of one of ordinary skill in the art.

According to some embodiments, the methods described herein may result in an increase in the oil extraction yield of the microorganism. For example, the method may result in an increase in the oil extraction yield of the microorganism of at least about 10 weight percent.

### Example 1

Fermentation of an oleaginous yeast strain using a complex sugar source that included sugar juice yielded unpasteurized whole broth for further processing. The whole broth was pasteurized by heating in a vessel from 27° C. to 80° C. in 30 minutes held at 80° C. for 3 hours. The pasteurized broth showed a fatty acid extractability (FAE) of 16.8%. No oil phase was recoverable upon centrifugation of the pasteurized un-lysed broth in a bench centrifuge at 4500 rpm (4000 g) for 5 minutes.

An aliquot of the pasteurized broth was pre-treated for 8 hours at 106° C. in a 20 L jacketed tank fitted with 2 Rushton impellers. The temperature increase from 26° C. to 106° C. occurred over about 45 minutes, at a rate of about 1.8° C./min. The pre-treated broth showed an increase in fatty acid extractability (80.75%). No oil phase was recoverable upon centrifugation of the pasteurized un-lysed broth in a bench centrifuge at 4500 rpm for 5 minutes. Results are presented in Table 2.

TABLE 2
Aqueous Extraction Conditions and Results of Example 1

| | Pasteuriz. | | Pre-treatment | | | Milling | | | Coalesc. | | Centrifug. | | | |
|---|---|---|---|---|---|---|---|---|---|---|---|---|---|---|
| | conditions | | conditions | | | conditions | | | Conditions | | conditions | | | |
| Fermentation | T | T | T | T | | Flow rate, | No. of | | | Time | Bench | | Extract | % |
| ID | hr | ° C. | hr | ° C. | pH | ml/m | Passes. | FAE | pH | hr | Centrifugation | | yield | FFA |
| N7 1022m12, | 3 | 80 | — | — | — | — | — | 16.84 | — | — | — | — | No | — |
| pH 6.31 | | | | | | | | | | | | | free | |
| Whole broth | | | | | | | | | | | | | oil | |
| fat % = 38.03, | 3 | 80 | — | — | — | 380 | 1 | 81.02 | — | — | — | — | — | — |
| dry basis | 3 | 80 | — | — | — | 380 | 2 | 92.31 | — | — | — | — | — | — |
| | 3 | 80 | — | — | — | 80 | 1 | 91.51 | — | — | — | — | — | — |
| | 3 | 80 | — | — | — | 80 | 2 | 94.8 | 4 | >40 h | 4000 g | 5 min | 69.89 | 1.22 |
| | 3 | 80 | 8 | 106 | 5.5 | — | — | 80.76 | — | — | — | — | No | — |
| | | | | | | | | | | | | | free | |
| | | | | | | | | | | | | | oil | |
| | 3 | 80 | 8 | 106 | 5.5 | 380 | 1 | 95.31 | — | — | — | — | — | — |
| | 3 | 80 | 8 | 106 | 5.5 | 380 | 2 | 95.53 | 5.5 | <16 h | 4000 g | 5 min | 79.21 | 1.2 |
| | 3 | 80 | 8 | 106 | 5.5 | 380 | 2 | 95.53 | 4 | <16 h | 4000 g | 5 min | 84.1 | 1.3 |

The pasteurized and pre-treated broth were each lysed at varying flow rates, 80 ml/minute or 380 ml/minute for 1 or 2 passes in a KDL Pilot bead-mill (1.4 L vessel filled to 85% fill volume with 0.5 mm silica-zirconia media). The pretreated broth exceeded the fatty acid extractability (FAE) of the pasteurized broth with minimal residence time in the mill. The extractability of pretreated broth lysed for 1 pass at the highest speed (380 ml/min) was comparable to the extractability of the pasteurized broth when processed at lowest speed (80 ml/min) for multiple passes.

Sample (200-300 g) of pasteurized or pretreated lysed broth with fatty acid extractability ˜95% was adjusted to pH 4 using 3N sulfuric acid. The sample was coalesced in batch mode under reflux (500 ml Erlenmeyer flask with stir-bar). Coalescence was monitored by centrifugation of 15-50 ml aliquot at 4500 rpm (4000 g) for 5 min in a bench centrifuge.

Coalesced broth upon centrifugation showed a distinct separate oil layer with a lower layer comprising spent broth. Coalescence of the pre-treated lysed broth was completed within 16 hours. The pasteurized lysed broth required over 40 hours to coalesce.

The oil layer was recovered from the top of the centrifuged tubes to estimate extraction yield. The extraction yield for the pre-treated broth was 84.1% while that of the pasteurized broth was 69.9%.

The oil quality is commonly determined by level of free fatty acids (FFA) via titration. The free fatty acid level in crude oil recovered from both the pasteurized broth and pre-treated broth were similar. (1.2-1.3%)

### Example 2

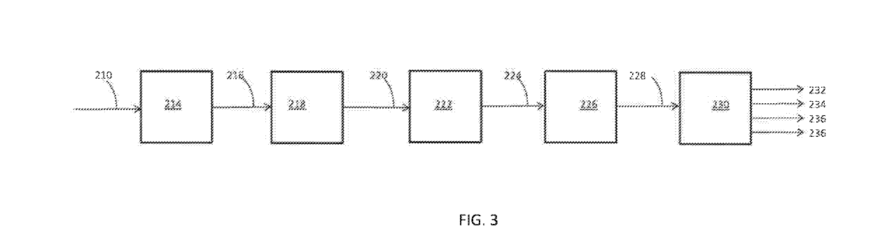

Fermentation of an oleaginous yeast strain using a complex sugar source that included sugar juice yielded unpasteurized whole broth for further processing. A flow diagram of the process of this example is illustrated in FIG. 3. As shown in FIG. 3, unpasteurized whole broth 210 was extracted using a protocol that included pasteurization of the broth 210 in an agitated jacketed vessel 214 at 80° C. for 3 hours. The pasteurized broth 216 was then adjusted to pH 4 using sulfuric acid and subjected to a pre-treatment phase 218 at 121° C., pressure of 30 psi (15 psig) for 8 hours. A temperature ramp of 1.8° C./min was used for the heating of broth; broth was cooled at a rate of 0.23° C./min. The pretreated broth 220 was then subjected to a lysing phase 222 using a bead-mill at 200 ml/min for 2 passes. The lysed broth 224 was then subjected to a coalescence phase 226 in a well-agitated tank at 90° C., 70% moisture. The coalesced broth 228 was then subjected to a solid-liquid separation phase 230 in which the coalesced broth 228 was centrifuged through two-phase and three-phase centrifuges to recover the crude oil 232. The process also yielded a spent broth phase, which was separated out into a spent heavy phase 234, and multiple assorted solid streams 236.

The concentration of metals in a sample of the unpasteurized whole broth, in the recovered oil and in each of the exit streams including the spent heavy phase and the solids was analyzed by ICP analysis. The ratio of concentration of metals in unpasteurized whole broth and in the recovered crude oil shows that the starting whole broth had at least twice the concentration of metals (excluding Si and Cu) than the oil recovered from the process. The crude oil recovered from the process was significantly depleted of Na, Mg, P, K, Ca, Mn, Fe, and Zn as compared to the whole fermentation broth.

The crude oil recovered from the extraction process was also significantly depleted of Na, Mg, P, K, Ca, Mn, Fe, and Zn compared to the other streams that exited the process such as the spent heavy phase and the solids following extraction.

TABLE 3
Comparison of Concentrations of Metals in Whole Broth to Crude Oil Recovered from
Aqueous Extraction Process

| | Na | Mg | Si | P | K | Ca | Mn | Fe | Cu | Zn | Mo |
|---|---|---|---|---|---|---|---|---|---|---|---|
| Sample Name | [mg/L] | [mg/L] | [mg/L] | [mg/L] | [mg/L] | [mg/L] | [mg/L] | [mg/L] | [mg/L] | [mg/L] | [mg/L] |
| Unpasteurized whole broth | 1658 | 486 | 93 | 389 | 7342 | 1387 | 2 | 66 | 0 | 22 | 0 |
| (MN082713-1 N2 | | | | | | | | | | | |
| 0617m13) | | | | | | | | | | | |
| Spent heavy phase | 1603 | 506 | 83 | 352 | 6807 | 1003 | 5 | 141 | 0 | 20 | 2 |
| (MN082713-2 N2 | | | | | | | | | | | |
| 0617m13) | | | | | | | | | | | |
| Solids from Centrifuge (#4) | 940 | 312 | 36 | 337 | 4238 | 2651 | 6 | 273 | 4 | 20 | 11 |
| (MN082713-3 N2 | | | | | | | | | | | |
| 0617m13) | | | | | | | | | | | |
| Solids from Centrifuge (#5) | 1474 | 448 | 60 | 425 | 6481 | 2292 | 6 | 257 | 4 | 25 | 8 |
| (MN082713-4 N2 | | | | | | | | | | | |
| 0617m13) | | | | | | | | | | | |
| Crude-oil | 71 | 26 | 71 | 60 | 283 | 53 | 0 | 8 | 0 | 1 | 0 |
| (SR100113-13 N2 | | | | | | | | | | | |
| 0617m13) | | | | | | | | | | | |
| Concentration Ratio, | 23 | 19 | 1 | 7 | 26 | 26 | 9 | 9 | 1 | 19 | 2 |
| Unpasteurized whole broth | | | | | | | | | | | |
| to Crude oil | | | | | | | | | | | |
| Concentration Ratio, Spent | 23 | 20 | 1 | 6 | 24 | 19 | 17 | 18 | 1 | 17 | 10 |
| heavy phase (liquid) to | | | | | | | | | | | |
| Crude oil | | | | | | | | | | | |
| Concentration Ratio, solids | 13 | 12 | 1 | 6 | 15 | 50 | 21 | 36 | 10 | 18 | 69 |
| from centrifuge (#4) to | | | | | | | | | | | |
| Crude oil | | | | | | | | | | | |
| Concentration Ratio, solids | 21 | 17 | 1 | 7 | 23 | 3 | 22 | 34 | 9 | 22 | 52 |
| from centrifuge (#5) to | | | | | | | | | | | |
| Crude oil | | | | | | | | | | | |

### Example 3

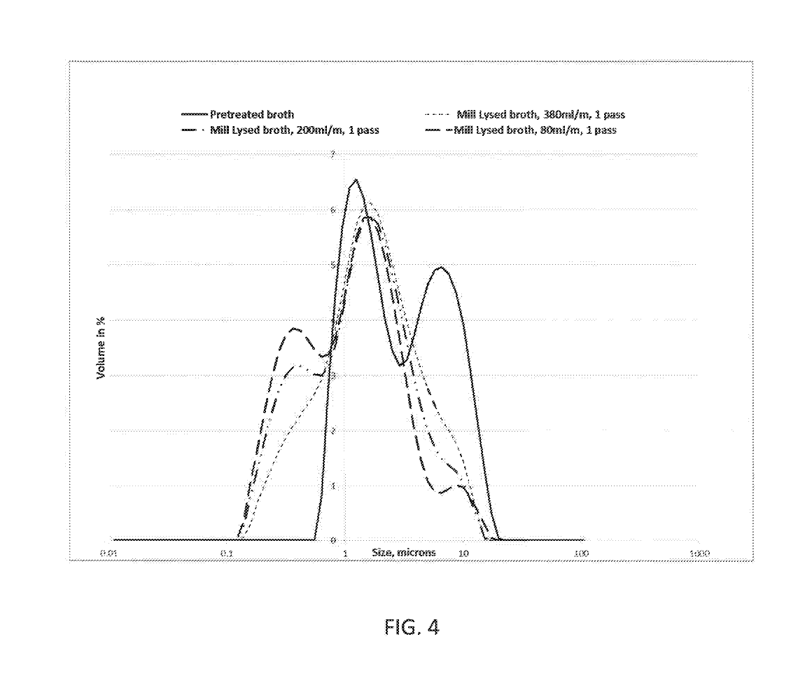

Whole fermentation broth of an oleaginous yeast strain was heated to 121° C. in an agitated vessel for 4 hours. The broth was thereafter cooled to 60° C. and lysed through a bead mill (KDL Pilot, Glen Mills, N.J.) run at 3 different flow rates respectively (380 ml/min, 200 ml/min, and 80 ml/min) to release intracellular oil product. Particle size distribution of released oil and cell debris following lysis in the mill is shown in FIG. 4. All measurable volume of lysed cells and oil droplets exceeds 0.1 microns, indicating to the potential to use processes such as centrifugation to separate an oil and solids phase upon further processing.

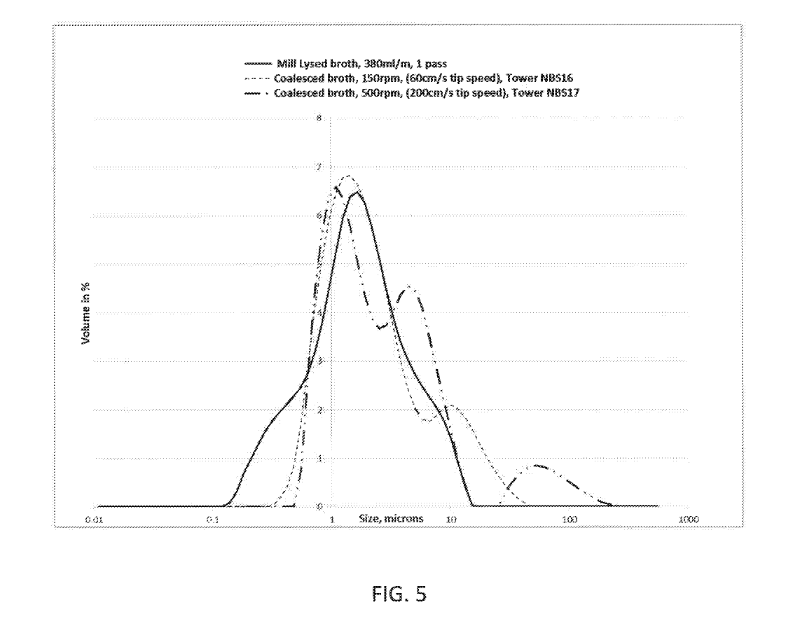

The oil product in the oil fraction can be recovered by mixing at 80° C. or higher temperatures. 5 L each of broth lysed at 380 ml/m was placed in a vessel mixed with two 3-inch (7.62 cm) Rushton impellers. The lysed broth was mixed at agitator speed of 150 rpm (tip speed=60 cm/s, tower NBS16) or at an agitator speed of 500 rpm (tip speed=200 cm/s, Tower NBS17). The distribution of oil and cell debris at the end of 6 hours of mixing is shown in FIG. 5. Product from the vessel at 500 rpm, tip speed of 200 cm/s, showed a distinct separate oil phase upon centrifugation at 4500 rpm (4000 g) for 5 minutes in a bench top centrifuge. Product from the vessel at 150 rpm, tip speed 60 cm/s, show no free oil and demonstrated an emulsion phase upon centrifugation.

It will be apparent to those skilled in the art that various modifications and variations can be made in the disclosed structures and methods without departing from the scope or spirit of the invention. Particularly, descriptions of any one embodiment can be freely combined with descriptions or other embodiments to result in combinations and/or variations of two or more elements or limitations. Other embodiments of the invention will be apparent to those skilled in the art from consideration of the specification and practice of the invention disclosed herein. It is intended that the specification and examples be considered exemplary only, with a true scope and spirit of the invention being indicated by the following claims.

1. A method of extracting lipids suitable in production of biofuels from a whole fermentation broth, comprising:
pre-treating the whole fermentation broth by heating the broth to a temperature between about 90° C. and about 150° C., or between about 100° C. and about 150° C., or between about 110° C. and about 150° C., or between about 120° C. and about 150° C., or between about 130° C. and about 150° C., wherein the broth contains oleaginous microorganisms; and
subsequently extracting a product from the oleaginous microorganisms.

2. The method of claim 1, wherein the pre-treating comprises heating the whole fermentation broth for about 30 minutes to about 18 hours, or more than 3 hours to about 18 hours, or more than 3 hours to about 8 hours.

3. The method of claim 1, wherein the pre-treating comprises heating the whole fermentation broth containing the oleaginous microorganisms from 45° C. to 80° C. in less than 60 minutes.

4. The method of claim 1, wherein the heating f the whole fermentation broth is done at an average rate between about 0.1 and about 80 degrees Celsius per minute.

5. The method of claim 1, further comprising adjusting pH of the whole fermentation broth by adding either an acid or a base.

6. The method of claim 1, further comprising cooling the whole fermentation broth to greater than about 60° C., or greater than about 70° C., or greater than about 80° C., or greater than about 85° C., or greater than about 90° C. to allow further isothermal processing.

7. The method of claim 6, wherein the further isothermal processing comprises applying mechanical disruption.

8. The method of claim 1, comprising cooling the pre-treated whole fermentation broth at an average rate between about 0.2 and about 80 degrees Celsius per minute.

9. The method of claim 1, further comprising drying the whole fermentation broth, following the heating.

10. The method of claim 1, further comprising agitating the whole fermentation broth at an impeller tip speed between about 10 cm per second and about 240 cm per second during the pre-treating.

11. The method of claim 1, further comprising maintaining a pressure between about 10 psi and about 150 psi, or between about 20 psi and about 150 psi, or between about 30 psi and about 150 psi, or between about 50 psi and about 150 psi in a system containing the whole fermentation broth during the pre-treating.

12. The method of claim 1, wherein during the pre-treating salts are present in a system containing the whole fermentation broth, resulting in an ionic strength between about 0.01 M and about 2 M in the system.

13. The method of claim 1, further comprising subjecting the oleaginous microorganisms to lysis, resulting in a droplet and debris particle size distribution in which at least 80% of a volume of released product oil droplets and debris have a size greater than 0.1 um in diameter.

14. The method of claim 1, further comprising subjecting the oleaginous microorganisms to lysis, resulting in a droplet and debris particle size distribution in which at least 95% of a volume of released product oil droplets and debris have a size greater than 0.1 um in diameter.

15. The method of claim 14, further comprising recovering the oil and cell debris droplets as a continuous phase by mixing at an impeller tip speed of greater than 120 cm/s.

16. The method of claim 1, wherein the pre-treated fermentation broth that has been through the pre-treatment may be coalesced within less than 8 hours by heating the fermentation broth for an additional 30 minutes to about 8 hours at over 90° C.

17. The method of claim 1, wherein the extraction process concentrates metals in the whole fermentation broth compared to the oil by a ratio of at least 2.

18. The method of claim 1, wherein a crude sugar source is used as a carbon source in the fermentation and results in extraction of a crude oil that is lower in metals and inorganic elements in comparison to extraction techniques that utilize whole dried biomass and/or solvents to recover crude oil.

19. The method of claim 1, wherein the whole fermentation broth comprises a crude sugar source associated with salts and ions at a concentration >0.05 g/L.

20. The method of claim 19, wherein the salts and ions are selected from the group consisting of Na, K, Ca, Mg, Zn, and chlorides, sulfates, phosphates, nitrates, and combinations thereof.

21. The method of claim 19, wherein the salts and ions comprise potassium, calcium, or combinations thereof.

22. The method of claim 19, wherein the salts and ions accumulate to a concentration of 0.5 to 40 g/L.

23. The method of claim 19, wherein the salts and ions comprise a higher concentration of potassium than sodium.

24. The method of claim 19, wherein the salts and ions comprise calcium at a concentration greater than 1 g/L.

25. The method of claim 19, wherein the salts and ions comprise potassium at a concentration greater than 2.5 g/L.

26. The method of claim 19, wherein the salts and ions promote coalescence when the product is released from the oleaginous microorganisms.

27. The method of claim 26, wherein the fermentation broth comprises a concentration of salts and ions of 0.5 to 40 g/L during the coalescence.

28. The method of claim 26, wherein the coalescence results in a coalesced lipid particle size distribution in which at least 80% of a volume of coalesced lipids have a size greater than 40 um in diameter.

29. The method of claim 26, wherein the coalescence results in a coalesced lipid particle size distribution in which at least 95% of a volume of coalesced lipids have a size greater than 40 um in diameter.

30. The method of claim 1, further comprising depressurizing the fermentation broth, following the heating, and cooling the whole fermentation broth to concentrate solids in the broth prior to further processing.

31. The method of claim 1, further comprising adding a solvent to the dry cells or lysed fermentation broth, following the heating, to form a mixture.

32. The method of claim 31, wherein the solvent is selected from the group consisting of hexane, dodecane, decane, diesel, alcohols, and combinations thereof.

33. The method of claim 31, further comprising agitating the mixture of the lysed fermentation broth and the solvent to contact and extract oil from the oleaginous microorganisms.

34. The method of claim 33, further comprising separating the solvent and the oil from the lysed fermentation broth.

35. The method of claim 34, comprising using a centrifuge to separate the solvent and the oil from the lysed fermentation broth.

36. The method of claim 34, further comprising reacting the solvent and the oil to convert at least a portion of the oil into a fuel component.

37. The method of claim 36, further comprising converting the solvent and a remainder of the oil into a fuel comprising a biofuel.

38. The method of claim 34, further comprising using the fermentation broth from which oil was separated as fertilizer for crops, animal feed, yeast extract, yeast hydrolysate, or a source of carbon/nutrients.

39. The method of claim 1, wherein the whole fermentation broth containing the oleaginous microorganisms comprises a sugar feedstock.

40. The method of claim 39, wherein the whole fermentation broth containing the oleaginous microorganisms and the sugar feedstock comprises about 50 to about 250 grams of lipid per liter of fermenter broth, about 0 to about 50 grams of sugar per liter of fermenter broth, about 0 to about 40 grams of salt per liter of fermenter broth, and about 10 to about 100 grams of lipid-free dry biomass per liter of fermenter broth.

41. The method of claim 1, wherein the oleaginous microorganisms comprise at least 40% by weight fat.

42. The method claim 1, further comprising pasteurizing a whole fermentation broth containing the oleaginous microorganisms during pre-treating.

43. The method of claim 42, comprising pasteurizing the whole fermentation broth by heating the whole fermentation broth to about 40° C. to about 80° C. for between about 1 minute and about 3 hours.

44. The method of claim 43, further comprising holding the whole fermentation broth at a temperature between about 90° C. and about 150° C., or between about 100° C. and about 150° C., or between about 110° C. and about 150° C., or between about 120° C. and about 150° C., or between about 130° C. and about 150° C. for about 30 minutes to about 18 hours, or more than 3 hours to about 18 hours, or more than 3 hours to about 8 hours.

45. The method of claim 44, further comprising stirring the whole fermentation broth during the heating interval.

46. The method of claim 44, further comprising adding an acid to the whole fermentation broth.

47. The method of claim 44, further comprising adding a base to the whole fermentation broth.

48. The method of claim 44, further comprising passing the whole fermentation broth through a bead mill, a homogenizer, an orifice plate, a high-shear mixer, a press, an extruder, pressure disruption, wet milling, dry milling, or other shear or mechanical disruption device at least once.

49. The method of claim 48, comprising passing the whole fermentation broth through a bead mill, a homogenizer, an orifice plate, a high-shear mixer, a press, an extruder, pressure disruption, wet milling, dry milling, or other shear or mechanical disruption device at least twice.

50. The method of claim 48, further comprising stirring the lysed fermentation broth in a vessel at about 70° C. to about 100° C. for about 1 to about 60 hours.

51. The method of claim 50, further comprising adding a salt to the lysed fermentation broth in the vessel.

52. The method of claim 51, comprising adding up to about 2% by weight of the salt.

53. The method of claim 51, wherein the salt is NaCl, KCl, K₂SO₄, Na₂SO₄, or derived from a combination of at least one NaOH and KOH plus H₂SO₄.

54. The method of claim 50, further comprising adding a base to adjust a pH of the lysed fermentation broth in the vessel to between about 3 and about 11.

55. The method of claim 51, further comprising separating oil that is less than 20% free fatty acids from the whole fermentation broth through centrifugation.

56. The method of any claim 1, wherein the oleaginous microorganisms are oleaginous yeast cells.

57. The method of claim 1, further comprising using a combination of enzymes to break down oleaginous cell walls of the oleaginous microorganisms, wherein the enzymes include amylase, 1-4 mannosidase, and 1-3 mannosidase.

58. The method of claim 57, wherein the combination of enzymes further comprises at least one auxiliary enzyme selected from the group consisting of sulfatase, protease, and chitinase.

59. The method of claim 57, wherein the amylase is specific for alpha 1-4 linked glucose.

60. The method of claim 57, wherein the combination of enzymes comprises between about 5% and about 30% by weight amylase.

61. The method of claim 57, wherein the combination of enzymes comprises between about 5% and about 45% by weight 1-4 mannosidase.

62. The method of claim 57, wherein the combination of enzymes comprises between about 5% and about 45% by weight 1-3 mannosidase.

63. The method of claim 57, further comprising harvesting intracellular metabolites from the oleaginous cell walls after breaking down the oleaginous cell walls.

64. The method of claim 63, wherein the intracellular metabolites comprise lipids.

65. The method of claim 63, further comprising converting the intracellular metabolites into biofuel.

66. The method of claim 63, further comprising recycling aqueous extraction effluent remaining after harvesting the intracellular metabolites.

67. The method of claim 66, further comprising using the recycled extraction water as imbibition water for washing a process feedstock to extract sugar.

68-112. (canceled)
